(19) United States
(12) Patent Application Publication
(10) Pub. No.: US 2009/0193680 A1
(43) Pub. Date: Aug. 6, 2009

(54) REMOTE CONTROL PULSE MASSAGE SHOE

(76) Inventor: CHUNG CHI PANG, Kowloon (HK)

Correspondence Address: JOHN ALEXANDER GALBREATH, 2516 CHESTNUT WOODS CT, REISTERSTOWN, MD, 21136, US

(21) Appl. No.: 12/235,533

(22) Filed: Sep. 22, 2008

(30) Foreign Application Priority Data
Feb. 4, 2008 (CN) 200820092257.4

(51) Int. Cl. A43B 13/38; A61F 5/14

(52) U.S. Cl. 36/43; 36/141; 601/27

(57) ABSTRACT

A remote control pulse current massage shoe having a shoe sole, a shoe insole, and vamp connected with the shoe sole. The shoe insole is equipped with insulated positive pole and negative pole. The shoe sole is equipped with a pulse generator to generate pulse current. A remote control signal receiver is used to receive control signal from the outside remote controller for controlling the switch of the pulse generator, voltage stiffness and frequency. The remote control signal receiver is connected with the pulse signal generator. The device permits easy adjustment and simple operation on the pulse current massage shoe by users or other people.

### CROSS-REFERENCE TO RELATED APPLICATION

The present patent application is based on and claims priority from Chinese Application 200820092257.4 filed Feb. 4, 2008.

### BACKGROUND OF THE INVENTION

The invention relates generally to a remote control pulse current massage shoe, and in particular to a pulse current massage shoe that can be remotely controlled to have a pulse current pass through the foot of the user.

There are many meridian-collaterals and acupoints on the human foot. Foot massage can enhance physiological function of various organs in the digestive system, respiratory system, circulation system, and endocrine system for achieving the goal of curing diseases and providing good healthcare.

Chinese Patent Application 200610033971.1 introduces a pulse current massage shoe, comprising: a shoe sole; a shoe insole placed on the shoe sole and vamp connected with the shoe sole; the shoe insole is installed with a positive pole and a negative pole; the shoe sole is equipped with a pulse generator to generate pulse current; the generator includes a positive terminal connected with positive pole on the shoe insole, and a negative terminal connected with the negative pole on the shoe insole; the positive pole, negative pole and human foot connect with the pulse generator forming a loop to have pulse current pass through the foot for stimulating and massaging the foot.

All operation of the switch, mode selection and intensity selection on the pulse current massage shoe is required to be achieved by pressing the button equipped on the shoe sole. In using the shoe, when it is desired to adjust the massage intensity, the mode of usage or to power on/off the shoe, the user is required to bend down to touch the shoe, which is inconvenient for the elderly or people with disabilities.

Aiming at the shortcomings in the above technology, the present invention is designed to solve the technical problem for providing a remote control pulse current massage shoe which enables convenient operation.

### SUMMARY OF THE INVENTION

The present invention provides a remote control pulse current massage shoe, comprising: a shoe sole; a shoe insole placed on said shoe sole; a vamp connected with said shoe sole; said shoe insole is equipped with an insulated positive pole and an insulated negative pole; said shoe sole is equipped with a pulse generator to generate a pulse current; said generator includes a positive terminal connected with said positive electrode on said shoe insole; said generator includes a negative terminal connected with said negative pole on said shoe insole; said positive pole, said negative pole, and a foot of a human body connect with said pulse generator forming a loop; a remote control signal receiver is connected with said pulse signal generator; and said remote control signal receiver is used to receive control signal from an outside remote controller for controlling switching ON and OFF of said pulse generator, and for controlling voltage magnitude and frequency of said pulse current.

The technical solution adopted to solve the technical problem resides in: forming a remote control pulse current massage shoe, comprising: a shoe sole; a shoe insole placed on the shoe sole; a vamp connected with the shoe sole; the shoe insole is installed with positive pole and negative pole; the shoe sole is equipped with a pulse generator to generate pulse current; the generator includes a positive terminal connected with the positive electrode on the shoe insole, and a negative terminal connected with the negative electrode on the shoe insole; the positive pole, negative pole, and human foot connect with the pulse generator forming a loop; a remote control signal receiver is used to receive control signals from an outside remote controller for controlling the switch of the pulse generator, and the voltage magnitude and frequency; the remote control signal receiver is connected with the pulse signal generator.

In the remote control pulse current massage shoe of the invention, the pulse generator comprises of the pulse generation and output unit used for pulse current output; the control unit used for controlling voltage stiffness and frequency generated by the foresaid pulse generation unit; the power supply unit used for supplying power to the whole pulse generator; the foresaid pulse generation unit is connected with the control unit, and connected with the foresaid positive pole and negative pole by the foresaid positive terminal and negative terminal.

In the foresaid remote control pulse current massage shoe of the invention, the foresaid remote control signal receiver is connected with the foresaid control unit.

In the foresaid remote control pulse current massage shoe of the invention, it further comprises of a human body sensing unit used for detecting if human body contacts the foresaid shoe insole and discharges the electricity accumulated in the circuit in case of no contact of human body, this is connected with the foresaid control unit.

In the foresaid remote control pulse current massage shoe of the invention, it further comprises of a human-machine interaction response unit to generate response voice when receiving complete remote control signal used on the foresaid remote control signal receiver, this is connected with the foresaid control unit.

In the foresaid remote control pulse current massage shoe of the invention, the foresaid remote control receiver is an infrared remote receiver.

In the foresaid remote control pulse current massage shoe of the invention, the foresaid remote control receiver is installed in the inner side surface of the shoe sole.

In the foresaid remote control pulse current massage shoe of the invention, the sock made with conductive fiber is installed between the foresaid shoe insole and vamp.

In the foresaid remote control pulse current massage shoe of the invention, the positive and negative pole on the foresaid shoe insole are respectively connected with the two insulated and fixed magnetically conductive objects, the positive terminal and native terminal on the pulse generator are respectively connected with the two insulated conductive objects capable of pick-up by the magnetic conductive object on the shoe insole forming a loop with positive terminal, conductive object, magnetic conductive object, positive pole, human foot, negative pole, magnetic conductive object, conductive object, and negative terminal.

Application of the foresaid remote control pulse current massage shoe of the invention is with the following advantages: convenient adjustment on the remote control pulse current massage shoe by users or other people, and more simple operation.

### DESCRIPTION OF FIGURES

Further explanation to the invention will be stated below combining with the attached figures and the mode of carrying out the invention:

FIG. 1 shows the structure of the remote control pulse current massage shoe of the invention;

FIG. 2 is the frame diagram of the remote control pulse current massage shoe of the invention;

FIG. 3 is the circuit diagram of the remote control pulse current massage shoe of the invention;

FIG. 4 is the circuit diagram of the remote controller used for the remote control pulse current massage shoe of the invention;

FIG. 5 shows the structure of the shoe sole of the remote control pulse current massage shoe of the invention;

### MODE OF CARRYING OUT THE INVENTION

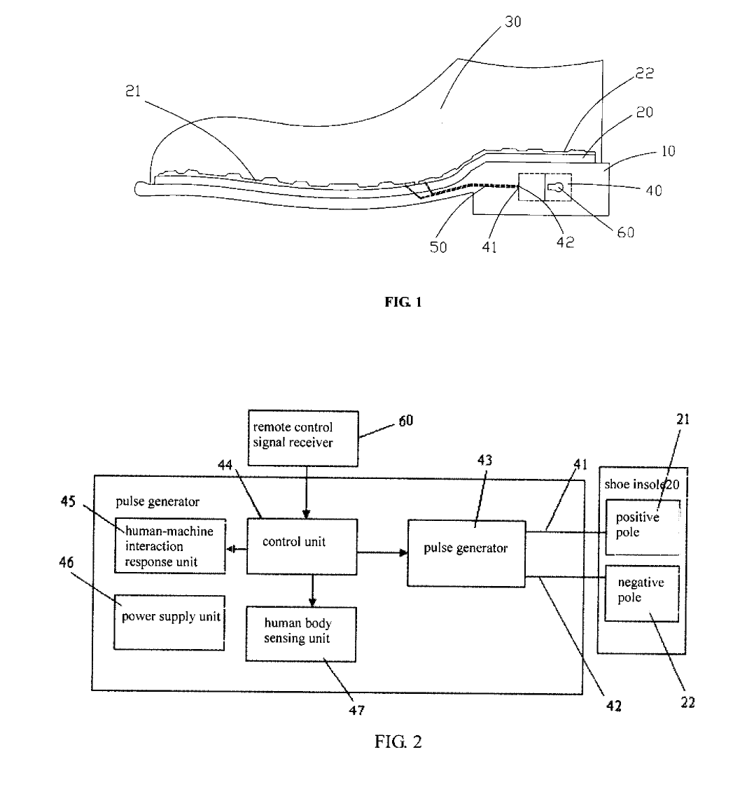

As shown in FIG. 1, the remote control pulse current massage shoe of the invention comprises a shoe sole 10, a shoe insole 20 placed on the shoe sole 10, a vamp 30 connected with the shoe sole 10, and a remote control signal receiver 60.

An insulated positive pole 21 and a negative pole 22 are provided on the shoe insole 20. The positive pole 21 is provided on the front part of the shoe insole 10, while the negative pole 22 is provided on the rear part the shoe insole 20. The positions of the positive pole 21 and the negative pole 22 can be exchanged, and located at any position at will. For better contact of a human foot with the positive pole 21 and the negative pole 22, a plurality of extrusions are formed on the shoe insole.

A pulse generator 40 is fixed in the shoe sole 10. The pulse generator 40 is equipped with a positive terminal 41 connected with positive pole 21, and a negative terminal 42 connected with the negative pole 22. In this embodiment, the positive terminal 42 is connected with the positive pole 21 by a cable 50, while the negative terminal 42 is connected with the negative pole 22 by cable 50. By foot contact, the positive pole 21 and the negative pole 22 are connected through to the pulse generator 40 forming a loop to have the pulse generator 40 generate pulse current passing through the human foot for stimulating and massaging foot and achieving easing effects for foot muscles. Moreover, the pulse generator 40 can be placed in a water-proof rubber cover, and all the connecting parts including cable 50 can be sealed with water-proof glue for a waterproof effect and prevention of current leakage.

The remote control signal receiver 60 is used to receive a remote control signal emitted from an outside remote controller, and to transmit the signal to the pulse generator 40. An infrared remote control receiver can be used. It can be installed in the inner side of the heel of the shoe sole 10, and other places convenient to receive the remote control signal as well. If the outside remote controller for cooperative use is an infrared remote controller, it transmits infrared data packets at a distance of up to five meters.

A sock made with conductive fiber can be provided between the shoe insole 22 and the vamp 30 for improving conductive performance and a more remarkable massage effect.

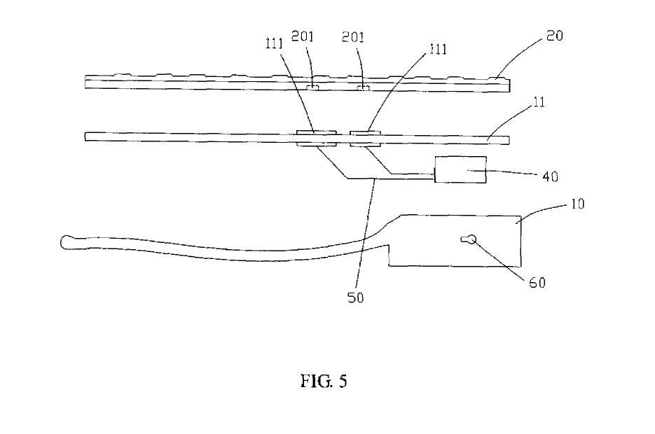

Due to the cable connection between shoe insole and shoe sole, it is not convenient for cleaning and replacement. As shown in FIG. 5, a secondary insole 11 made with insulating material can be installed between the shoe insole 20 and the shoe sole 10. Two insulated iron sheets 111 provided on the upper side of the secondary insole are respectively connected with the positive terminal 41 and the negative terminal 42; two insulated magnets 201 equipped on the back side of the secondary insole are respectively connected with the positive pole 21 and the negative pole 22.

The positive pole 21 connected with the magnet 201 is in the corresponding position with the positive terminal 41 connected with the iron sheet 111 for pick-up; while, the negative pole 22 connected with the magnet 201 is in the corresponding position with the negative terminal 42 connected with the iron sheet 111 for pick-up. The surface area of the iron sheet can a little larger than that of the magnet 201, pick-up can also be ensured in case of shoe insole moving in the shoe for forming a good conductive channel. During massage, the iron sheet 111 is pulled by the magnet 201 to form a current path from the pulse generator 40 and positive terminal 41 to positive pole 21 through the iron sheet 111 and magnet 201, then to negative pole through human foot, to negative terminal 42 through magnet 201 and iron sheet 111.

Certainly, the combination is not limited to the iron sheet 111 and magnets 201. As long as pick-up can be performed between the conductive materials, they can be used for connection.

It can also be free of the secondary insole, but directly to fix the iron sheet 111 on to the shoe sole 10. Because there is no cable on the shoe sole, cleaning and replacement can be more conveniently performed.

As shown in FIG. 2, the pulse generator 40 comprises of pulse generation and output unit 43, control unit 44 and power supply unit 46 to supply power to the whole pulse generator 40. The pulse generation and output unit 43 is connected with the control unit 44. The remote control signal receiver 60 is connected with the control signal input of the control unit 44 on the pulse generator 40. The remote control signal receiver 60 is used to receive the remote control information from the outside remote controller, and transmit the information to the control unit 44. The control unit 44 deals with the received information and transmits the control signal to the pulse generation unit 43. The pulse generation and output unit 43 generates pulse current according to the control signal from the foresaid control unit 44, and performs output when it forms a loop with human foot.

Power supply unit 46 is used to supply power to the whole pulse generator 40.

The pulse generator 40 further comprises of the human body sensing unit 47 and human-machine interaction response unit 45 connected with the control unit 44. The human body sensing unit 47 is used to detect if human body contacts with the massage shoe insole and inputs signal to the control unit 44. When the remote control signal receiver 60 receives the complete remote control signal, the human-machine interaction response unit 45 gives the response sound according to the directive from the control unit 44.

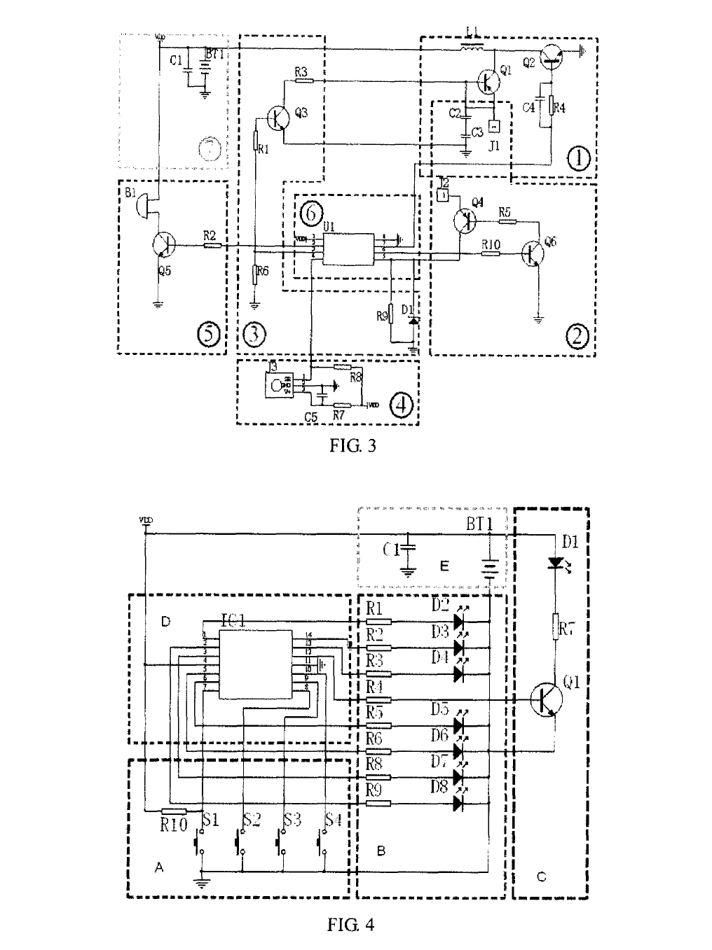

As shown in FIG. 3, in the circuit diagram of the pulse remote massage shoe of the invention, it comprises of boost circuit 1, massage pulse output circuit 2, human body sensing circuit 3, remote control signal receiver circuit 4, human-machine interaction response circuit 5, main control circuit 6 and power supply circuit 7.

The boost circuit 1 consists of electrical inductance L1 and NPN Audion Q2. The pulse train from Pin #7 of the MCU is the excitation signal. In case of forward pulse from Pin #7 of the MCU, Q2 is in close state, the current is full of magnetic energy after passing through L1 from power supply; In case of reverse pulse from Pin #7 of the MCU, Q2 is in cut-off state. At the time, the forward current passing through L1 suddenly disappears. The magnetic energy in magnetic core is in a hurry release to make L1 generate super strong reverse voltage. The high voltage accumulates in capacitor C2 and C3 through NPN Audion and Q1 commutation.

The massage pulse output circuit 2 consists of capacitor C2, C3, human body and Audion Q4. When excitation pulse is output from MCU, current is connected with the position with +2.4V of negative pole on diode D1 by human body and Q4, and forms a loop by connecting with the negative terminal of capacitor C3 through D1. It thus makes the most voltage in the capacitor go upon the human body. The muscle is forced to perform action due to the just-in-time defense function of the human body defense capacity.

The human body sensing circuit 3 consists of resistor R9 and diode D1. Human body sensing circuit is connected in the pulse output loop by series connection for detecting if human body contacts the shoe insole. If not, the electric energy accumulated in capacitor C2 and C3 will be discharged by NPN Audion Q3 to avoid a sense of electric shock while contacting with human body. The work principle is to determine whether there is current passing through the massage circuit by detecting the voltage on D1, and finds if the human body contacts the massage insole. No sudden intensity change of output is related with this circuit. During operation, if human body is apart from the massage insole, the intensity will be placed to be the weakest in case of detection by procedure. In case re-contact by human body, the output intensity increases steadily to the preset intensity for making people comfortable.

The remote control signal receiver circuit 4 consists of infrared receive assembly J3, resistor R8, R7 and capacitor C5. The function of it is to receive the infrared signal packet from the remote controller, and to provide to MCU for directive analysis after being converted into electrical signal. R8 is a pull-up resistor for enhancing the reliability of receiving the integrated output signal. R7 and C5 supplies reliable power for the receive assembly to achieve a stable signal receiving in case of unstable battery voltage.

The human-machine interaction response circuit 5 consists of a beeper B1, NPN Audion Q5 and resistor R2. When a complete signal is received, MCU controls to give a response sound.

The main control circuit 6 consists of a microcomputer controller U1 with the function of all operation on receiving part of the massager.

Power supply circuit 7 consists of battery BT1 and capacitor C1 with the function to supply power to the pulse generator. Function of C1 is for decoupling to avoid influence against normal work of U1 caused by unstable battery during operation of the boost circuit 1, and for improving the stability of the receiving part of the massager and security of U1 reset as well.

Work process of the circuit is as below:

When J3 receives the complete remote control signal, Pin #2 of the control chip U1 will output signal to make Q5 on-state, the control beeper B1 beeps. When J3 receives remote signal for power-on, the human body sensing circuit will be in standby state in case of no human body contact with the massage insole detected. At the moment, J1 is free of high voltage.

When the human body sensing circuit induces the contact of human body with the massage insole, the control chip U1 controls Q2 for on-state or cut-off by output of pulse train from Pin 37 according to the set mode for achieving periodic high voltage at two ends of C2 and C3. When human body contacts the massage insole, electrode 11 will be connected through with J2 by human body forming a current loop by C3, C2, human body, Q4 and D1. Due to the periodic high voltage at the two ends of C2 and C3, current in the loop is also the periodic current. Various massage modes and massage intensity can be realized by control on frequency of pulse train.

When changes for massage mode or massage intensity are needed, the remote controller is used to send remote directive for modification. This is a preset directive. When the infrared receiver J3 receives the directive from the remote controller, it transmits it to the control chip U1. U1 will decode the remote directive after receiving it, and then performs data distribution processing. After making analysis on the remote directive, the mode and intensity will be taken and saved according to the directive. If contact of human body with the massage insole is detected, pulse wave will be output according to the preset mode and intensity.

The maximum output transient voltage on the pulse current massage shoe of the invention is lower than 230V. The maximum lasting output high voltage time is less than 100 micro-seconds. The current passing through human body is lower than 80 μA. The overall power consumption is less than 70 mW.

As shown in FIG. 4, in the circuit diagram of the remoter controller used in the pulse current massage shoe of the invention, it comprises of human-machine interaction input keyboard A, human-machine interaction display B, information transmitting circuit C, main control circuit D of the remote controller and power supply circuit E.

The human-machine interaction input keyboard A consists of S1-S4. Power on/off and adjustment for massage mode and intensity of the pulse current massage shoe can be realized through the keys or key combination by users. This simulated signal is transmitted to the chip IC1.

The human-machine interaction display B consists of LED D2-D8, resistor R1-R3, R5-R9. The LED is used to display information in the remote controller to have the users know clearly the current status of the remote controller. Each LED can be defined for corresponding operation. When users click the key or key combination for operating the pulse current massage shoe, the corresponding LED lights up for more intuitionistic display of successful or failed operation. Functions of the resistor R1-R3, R5-R9 is to control current for protecting the LED.

The main control circuit D of the remote controller consists of the chip IC1. This part is the core of the remote controller for control on the all the operation of the remote controller. IC1 receives the simulated signal input by the human-machine interaction input keyboard A. After processing, it is converted to digital signal for output.

The information transmitting circuit C consists of LED D1, resistor R7, and NPN Audion Q1. It is receive the digital signal from IC 1 and transmits out. The pulse massage shoe can receive this signal.

The power supply circuit E consists of battery BT1 and capacitor C1 used for supplying power to the remote controller. C1 is for prevention from outside interfering signals against IC1, and providing reliable reset voltage for IC1. Generally, battery BT1 means two pieces of dry batteries.

When users operate the human-machine interaction input keyboard A, the human-machine interaction display B displays the current operation by users for mainly controlling the circuit D to receive this simulated signal. After information processing, the signal is converted into digital signal for information encoding and encapsulation. And then the data packet is transmitted through the information transmitting circuit C.

There has been described above only one possible embodiment of the invention, and the protection scope is not limited to this. Any changes or replacements easily got within the technical scope disclosed in this invention by technicians familiar with this technical field are in the scope of protection of this invention. Therefore, the protection scope of the invention is set forth in the following claims.

1. A remote control pulse current massage shoe, comprising:
a shoe sole;
a shoe insole placed on said shoe sole;
a vamp connected with said shoe sole;
said shoe insole is equipped with an insulated positive pole and an insulated negative pole;
said shoe sole is equipped with a pulse generator to generate a pulse current;
said generator includes a positive terminal connected with said positive electrode on said shoe insole;
said generator includes a negative terminal connected with said negative pole on said shoe insole;
said positive pole, said negative pole, and a foot of a human body connect with said pulse generator forming a loop;
a remote control signal receiver is connected with said pulse signal generator; and
said remote control signal receiver is used to receive control signal from an outside remote controller for controlling switching ON and OFF of said pulse generator, and for controlling voltage magnitude and frequency of said pulse current.

2. The remote control pulse current massage shoe according to claim 1, wherein said pulse generator includes:
a pulse generation and output unit used for pulse current output;
a control unit used for controlling voltage magnitude and frequency generated by said pulse generation unit; and
a power supply unit used for supplying power to the entire pulse generator;
and wherein said pulse generation unit is connected with said control unit, and is connected with said positive pole and said negative pole by said positive terminal and negative terminal.

3. The remote control pulse current massage shoe as set forth in claim 2, wherein:
said remote control signal receiver is connected with said control unit.

4. The remote control pulse current massage shoe as set forth in claim 3, including:
a human body sensing unit used for detecting if the human body contacts said shoe insole and discharges the electricity accumulated in the circuit in case of no contact by the human body; and
said human body sensing unit is connected with said control unit.

5. The remote control pulse current massage shoe as set forth in claim 3, including:
a human-machine interaction response unit to generate a response voice when receiving a complete remote control signal from said remote control signal receiver; and
said human-machine interaction response unit is connected with said control unit.

6. The remote control pulse current massage shoe as set forth in claim 3, wherein said remote control signal receiver is an infrared remote receiver.

7. The remote control pulse current massage shoe as set forth in claim 1, wherein:
said remote control signal receiver is installed in an inner side surface of said shoe sole.

8. The remote control pulse current massage shoe as set forth in claim 2, wherein:
said remote control signal receiver is installed in an inner side surface of said shoe sole.

9. The remote control pulse current massage shoe as set forth in claim 3, wherein:
said remote control signal receiver is installed in an inner side surface of said shoe sole.

10. The remote control pulse current massage shoe as set forth in claim 4, wherein:
said remote control signal receiver is installed in an inner side surface of said shoe sole.

11. The remote control pulse current massage shoe as set forth in claim 5, wherein:
said remote control signal receiver is installed in an inner side surface of said shoe sole.

12. The remote control pulse current massage shoe as set forth in claim 6, wherein:
said remote control signal receiver is installed in an inner side surface of said shoe sole.

13. The remote control pulse current massage shoe as set forth in claim 7, wherein:
a sock made with conductive fiber is installed between said shoe insole and said vamp.

14. The remote control pulse current massage shoe as set forth in claim 8, wherein:
a sock made with conductive fiber is installed between said shoe insole and said vamp.

15. The remote control pulse current massage shoe as set forth in claim 9, wherein:
a sock made with conductive fiber is installed between said shoe insole and said vamp.

16. The remote control pulse current massage shoe as set forth in claim 10, wherein:
a sock made with conductive fiber is installed between said shoe insole and said vamp.

17. The remote control pulse current massage shoe as set forth in claim 11, wherein:
a sock made with conductive fiber is installed between said shoe insole and said vamp.

18. The remote control pulse current massage shoe as set forth in claim 12, wherein:
a sock made with conductive fiber is installed between said shoe insole and said vamp

19. The remote control pulse current massage shoe as set forth in claim 7, wherein;
said positive and negative poles on said shoe insole are respectively connected with two insulated magnets; and
said positive and negative terminals of said pulse generator are respectively connected with two insulated conductive objects capable of being attracted by said magnets on said shoe insole forming a loop with said positive terminal, a first one of said magnets, a first one of said magnetic conductive objects, said positive pole, the human foot, said negative pole, a second one of said magnetic conductive objects, a second one of said magnets, and said negative terminal.

20. The remote control pulse current massage shoe as set forth in claim 8, wherein;
said positive and negative poles on said shoe insole are respectively connected with two insulated magnets; and
said positive and negative terminals of said pulse generator are respectively connected with two insulated conductive objects capable of being attracted by said magnets on said shoe insole forming a loop with said positive terminal, a first one of said magnets, a first one of said magnetic conductive objects, said positive pole, the human foot, said negative pole, a second one of said magnetic conductive objects, a second one of said magnets, and said negative terminal.
